(19) United States
(12) Patent Application Publication
(10) Pub. No.: US 2013/0112210 A1
(43) Pub. Date: May 9, 2013

(54) Oral Sleep Apnea Device

(71) Applicant: Ivan F. STEIN, Short Hills, NJ (US)

(72) Inventor: Ivan F. STEIN, Short Hills, NJ (US)

(21) Appl. No.: 13/668,877

(22) Filed: Nov. 5, 2012

Related U.S. Application Data
(60) Provisional application No. 61/555,164, filed on Nov. 3, 2011.

(51) Int. Cl. A61F 5/56

(52) U.S. Cl. 128/848

(57) ABSTRACT

An oral sleep apnea device is a dental appliance designed to advance the lower jaw of a user suffering from sleep apnea while reducing lingual discomfort and the risk of harmful tooth movement and rotation during use. The present invention accomplishes this through the use of a maxillary and a mandibular dental plate coupled to the upper and lower jaw, respectively, without direct contact with the incisal or labial surfaces of the anterior teeth. Both appliances comprise a lingual surface mount. The maxillary dental plate comprises an advancement mechanism. The advancement mechanism being operatively engaged to the mandibular dental plate repositions the mandibular plate to a forward location relative to the maxillary dental plate. During the repositioning of the mandibular dental plate the mandibular lingual surface mount distributes pressure evenly to the lingual surfaces on the lower jaw, reducing localized pressure points which can cause tooth movement.

The current application is a nonprovisional application and claims a priority to the U.S. provisional patent application Ser. No. 61/555,164 filed on Nov. 3, 2011. The current application is filed on Nov. 5, 2012 while Nov. 3, 2012 was on a weekend.

### FIELD OF THE INVENTION

The present invention relates generally to an oral airway management device, more specifically to a mandibular advancement splint intended for the treatment of obstructive sleep apnea.

### BACKGROUND OF THE INVENTION

Sleep apnea is a disorder that occurs when an individual sufferers from lengthy abnormal pauses in breathing during their normal sleep cycle. These abnormal pauses result in daytime sleepiness and fatigue normally associated with a significant level of sleep disturbance. In the most common form of sleep apnea, known as obstructive sleep apnea (OSA), breathing is frequently interrupted by a physical block to airflow despite respiratory efforts during sleep. Typically, OSA patients suffer from structural features that give rise to a narrowed airway which are exacerbated by low muscle tone and excessive soft tissue around the airway. The most common symptoms observed by individual's suffering from sleep apnea are fatigue and daytime sleepiness usually at levels associated with significant sleep disturbance. For those observing an OSA patient the symptoms are characterized by excessive and loud snoring with frequent sudden pauses. It is believed that sleep apnea affects one in every fifteen adults in the United States, but due to the plurality of factors that could potentially results in OSAs most common symptoms, current figures could be a very conservative estimate. While sleep apnea is commonly associated with sleep disturbance symptoms, it has also been linked with an increased risk of cardiovascular disease, stroke, high blood pressure, arrhythmias, diabetes, and sleep deprived driving accidents.

With the plurality of potentially life threatening conditions linked with sleep apnea, various treatment methods have been developed to alleviate and treat the condition. Current treatment methods developed for sleep apnea range from life style changes to surgical procedures. Of the methods currently available, mandibular advancement splints are one of the most recent and popular treatment options that have appeared within the last few years. Since their introduction, mandibular advancement splints have become one of the most commonly utilized treatment methods for OSA.

Mandibular advancement splints are removable oral appliances that are provided as an upper jaw and a lower jaw component. The mandibular advancement splints function by causing upper jaw to slightly misalign with the lower jaw upon engagement of the upper and lower jaw components. This misalignment between the two devices, results in the forward positioning of the lower jaw. By moving the lower jaw forward, the mandibular advancement splint is able to tighten the soft tissue and muscles localized around the airway alleviating of the constriction to the trachea. Additionally, the forward positioning of the lower jaw advances the user's tongue forward preventing occlusion to the trachea. This technique is commonly used to maintain and reopen an air way as part of the jaw thrust maneuver.

Although mandibular advancement splints provide a treatment option to user suffering from OSA, there are several short comings that have yet to be addressed. Some of the short comings associated with current mandibular advancement devices are related to their bulky and rigid construction. Typical mandibular advancement devices are provided as two moveable components that are affixed to one another. While having the upper and lower components affixed to one another provides structural support and stability, the affixed nature of the two components reduces necessary movement between the upper and lower jaw. This lack of movement can cause soreness and discomfort to a user over a period of time. Another disadvantage seen with the current mandibular advancement splints is related to size of the device with regards to the space within a user's mouth. Typical mandibular advancement devices are constructed in manner that covers the anterior teeth. This construction reduces the lingual space within a user's mouth causing persistent discomfort during use. Yet another disadvantage associated with current mandibular advancement splints is their potential to cause movement and shifting to a user's teeth which could potentially lead to tooth eruptions. Still yet another disadvantage shared by all of the previously mentioned shortcomings is the possibility of them resulting in a patient discontinuing their treatment protocol.

It is therefore the object of the present invention to provide a dental apparatus used in to treat obstructive sleep apnea that offers the user maximum comfort during use of the device reducing the risk of non-compliance due to discomfort. The present invention accomplishes this through the use of two independent appliances that are custom fitted to a user's upper and lower jaw. The upper and lower appliances are independent, and not affixed to each other, but tare able to limit excessive movement while allowing enough movement to prevent jaw pains associated with current rigid mandibular advancement devices. Additionally, the present invention leaves the incisal and labial surfaces of the anterior teeth uncovered creating increased lingual space and the ability for the user to close their lips during use. Furthermore the present invention is able to reduce movement and rotation of the user's teeth by providing a lingual splint which distributes pressure over a larger surface area. The combination of these features provides the present invention with maximum comfort to a user during use, reducing the risk of the non-compliance to the treatment protocol/

### BRIEF DESCRIPTIONS OF THE DRAWINGS

FIG. 1 is an expanded perspective view of the present invention displaying the relationship between the maxillary dental plate and the mandibular dental plate to the upper and lower jaw, respectively.

FIG. 2 is a frontal expanded perspective view of the present invention displaying the relationship between the maxillary dental plate and the mandibular dental plate to the upper and lower jaw, respectively.

FIG. 3 is a front view of the present invention displaying the maxillary dental plate and the mandibular dental plate being operatively engaged to each other for the purposes of performing a mandibular advancement.

FIG. 4 is a side perspective view of the present invention displaying the maxillary contact surface being advanced by the surface advancement mechanism against the mandibular contact surface.

FIG. 5 is a side view of the present invention advancing the mandibular dental plate.

FIG. 6 is a partial sectional view of the maxillary dental plate displaying the lingual splint and the pair of posterior teeth connection method as per the current embodiment of the present invention.

FIG. 7 is a partial sectional view of the mandibular dental plate displaying the lingual splint and the pair of posterior teeth connection method as per the current embodiment of the present invention.

### DETAIL DESCRIPTIONS OF THE INVENTION

All illustrations of the drawings are for the purpose of describing selected versions of the present invention and are not intended to limit the scope of the present invention.

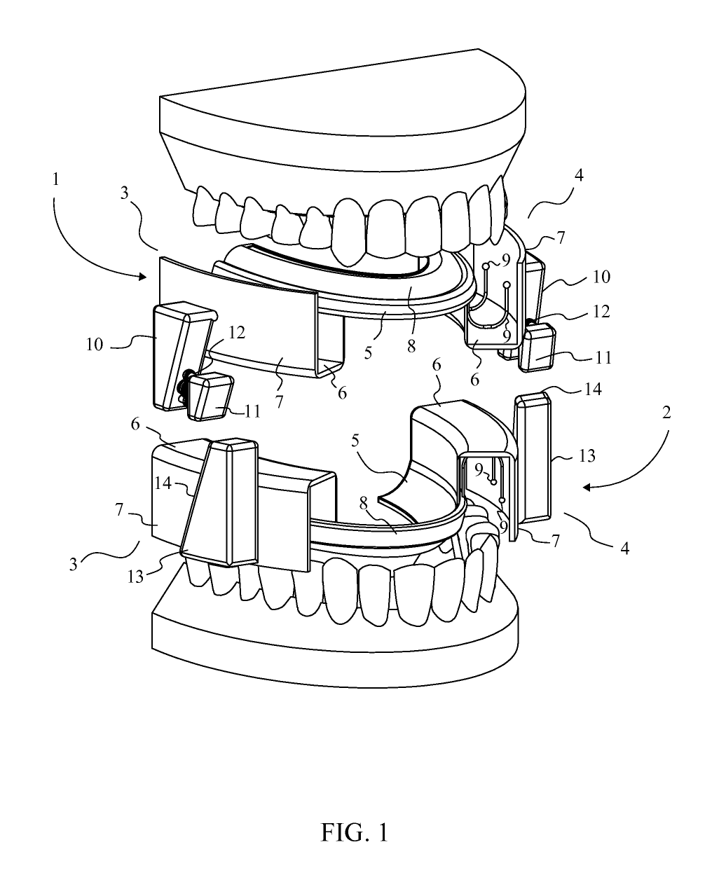

Referencing FIG. 1, the present invention is an oral sleep apnea device provided as a treatment option for patients suffering from obstructive sleep apnea (OSA). The device relieves the respiratory obstruction through the advancement of the lower jaw in a process commonly referred to as mandibular advancement. By performing a mandibular advancement, the present invention is able to move the tongue forward, preventing the tongue from occluding the tracheal entrance. The device additionally allows user's increased lingual comfort during use by eliminating direct contact with the incisal surfaces and the labial surfaces of the anterior teeth. The present invention comprises a maxillary dental plate 1 and a mandibular dental plate 2. The maxillary dental plate 1 and the mandibular dental plate 2 are both arcuately shaped. The arcuate shape for both the maxillary dental plate 1 and the mandibular dental plate 2 is provided by the position of a user's teeth on their upper and lower jaw, respectively. The maxillary dental plate 1 and the mandibular dental plate 2 are both bilaterally symmetrical along an imaginary median line which traverses centrally across a user's face. The maxillary dental plate 1 is positioned above the mandibular dental plate 2. The maxillary dental plate 1 is coupled to the upper jaw. The mandibular dental plate 2 is coupled to the lower jaw. The maxillary dental plate 1 is extendably coupled to the mandibular dental plate 2, wherein the coupling between maxillary dental plate 1 and the mandibular dental plate 2 allows the present invention to perform a mandibular advancement.

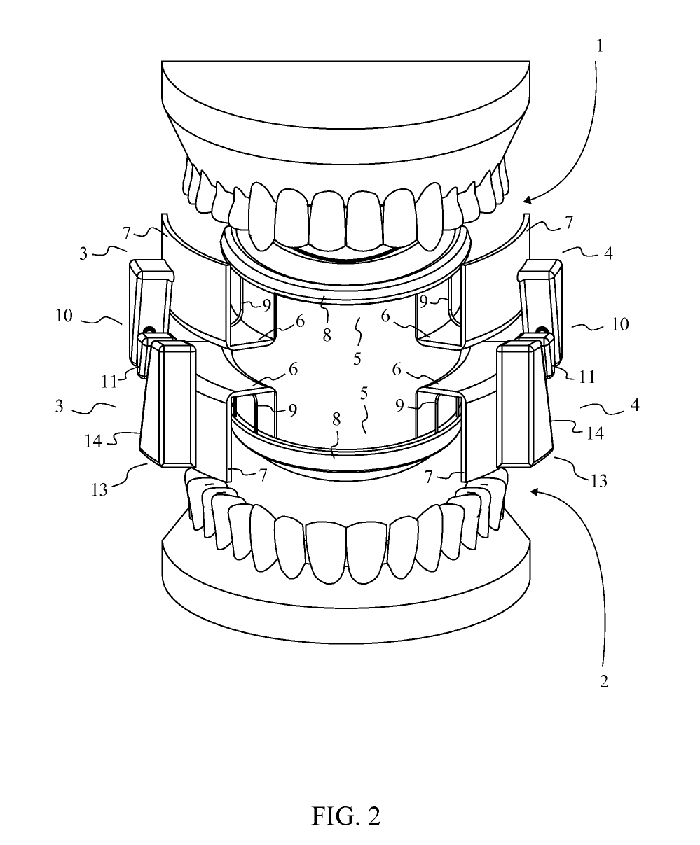

Referencing FIG. 2, both the maxillary dental plate 1 and the mandibular dental plate 2 comprise a first side section 3, a second side section 4, and a lingual surface mount 5. The lingual surface mount 5 is the point of contact between the device and the interior surfaces of the upper and lower jaw. The lingual surface mount 5 serves as part of a structural support system for the present invention, wherein the structural support system provides the present invention with the ability to be securely and comfortably couple to a user's jaw. The lingual surface mount 5 is positioned in close contact with the lingual surfaces of the anterior teeth, the posterior teeth, and the gums, wherein the anterior teeth comprise central incisors, lateral incisors, and cuspids (canine), and the posterior teeth comprise first and second biscuspids (first and second premolars) and first, second, and third molars. The lingual surface mount 5 of the present invention serves as an attachment point for the first side section 3 and the second side section 4. The first side section 3 and the second side section 4 are coupled along the lingual surface mount 5, wherein the first side section 3 and the second side section 4 are positioned opposite themselves across the median line.

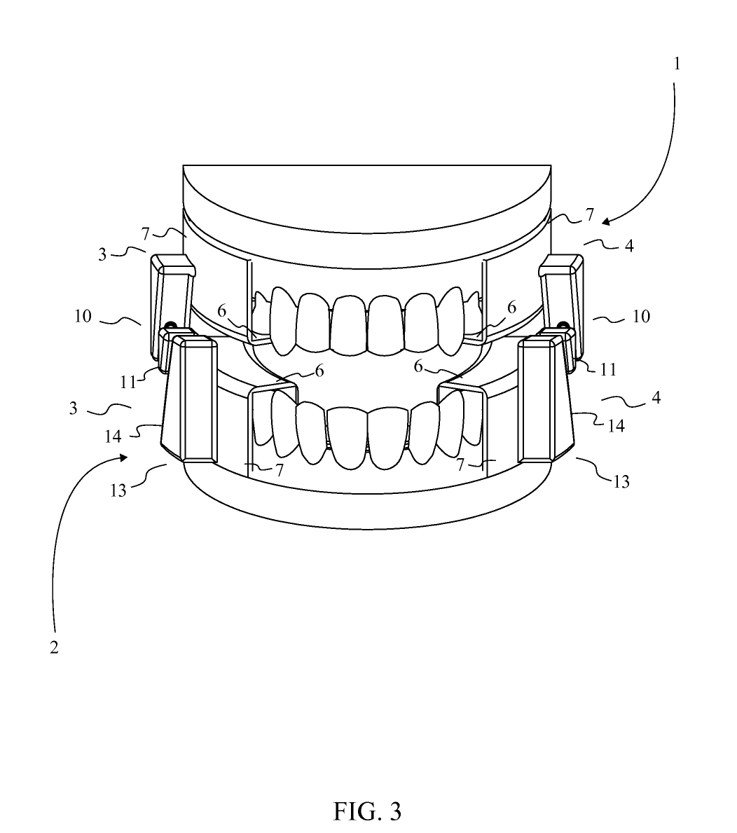

Referencing FIG. 2 and FIG. 3, both the first side section 3 and the second side section 4 comprise an occlusal surface 6 and a buccal surface 7. The occlusal surface 6 of the present invention contacts the chewing surfaces of the posterior teeth. The occlusal surface 6 serves as the attachment point for the first side section 3 and the second side section 4 to the lingual surface mount 5. The occlusal surface 6 traverses across the chewing surfaces of the posterior teeth from the lingual surface mount 5 to the facial surface. At the facial surface the occlusal surface 6 is found coupled to the buccal surface 7. The buccal surface 7 is positioned adjacent to the facial surfaces of the posterior teeth. The structural relationship formed between the buccal surface 7, the occlusal surface 6, and the lingual surface mount 5 provide the first and second section with a support structure and contacting surfaces between a user and the present invention.

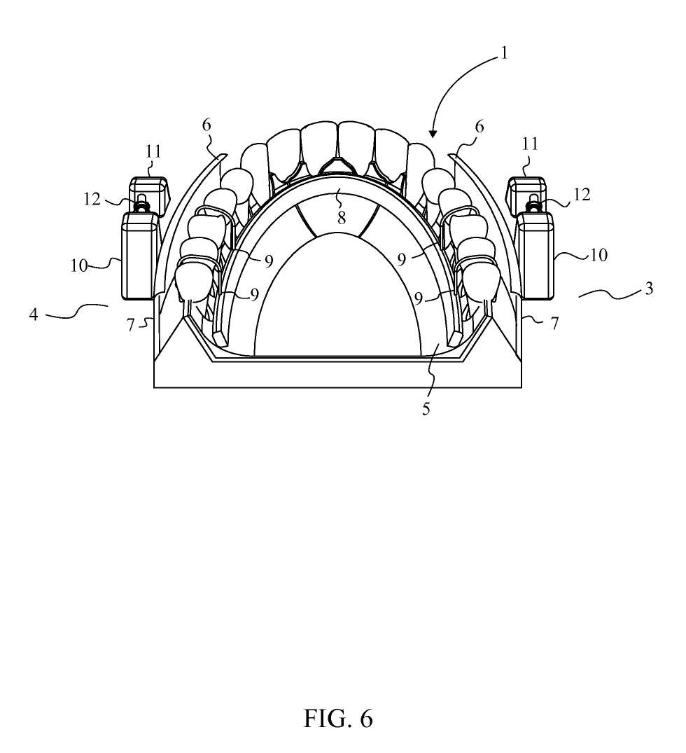

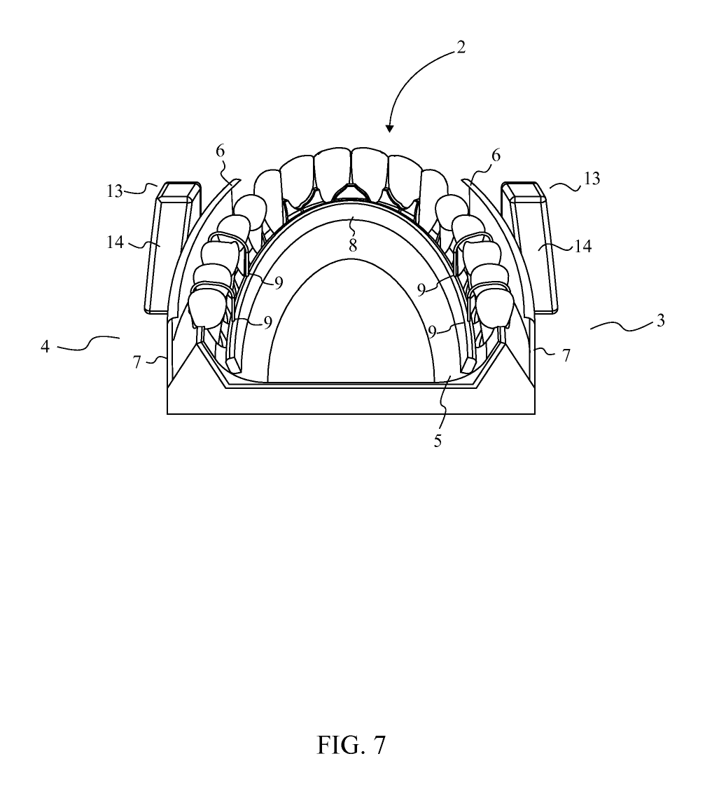

Referencing FIG. 6 and FIG. 7, lingual surface mount 5 further comprises a lingual splint 8 and a pair posterior teeth connection methods 9. The lingual splint 8 is found positioned within the lingual surface mount 5. The lingual splint 8 traverses through the lingual surface mount 5 along an arcuate path parallel to the positioning of the lingual surfaces of the anterior and posterior teeth against the lingual surface mount 5. The lingual splint 8 provides the lingual surface mount 5 with additional rigidity and support. The lingual splint 8 provides the present invention with improved durability. The lingual splint 8 is found coupled to the pair of posterior teeth connection methods 9. The pair of the posterior teeth connection methods 9 are positioned opposite one another across the median line. The pair of the posterior teeth connection methods 9 are found within the first side section 3 and the second side section 4, wherein the first side section 3 and the second side section 4 each house a posterior teeth connection method. The posterior teeth connection method provide the present invention with the ability to be removably coupled to the posterior teeth of a user's upper or lower jaw.

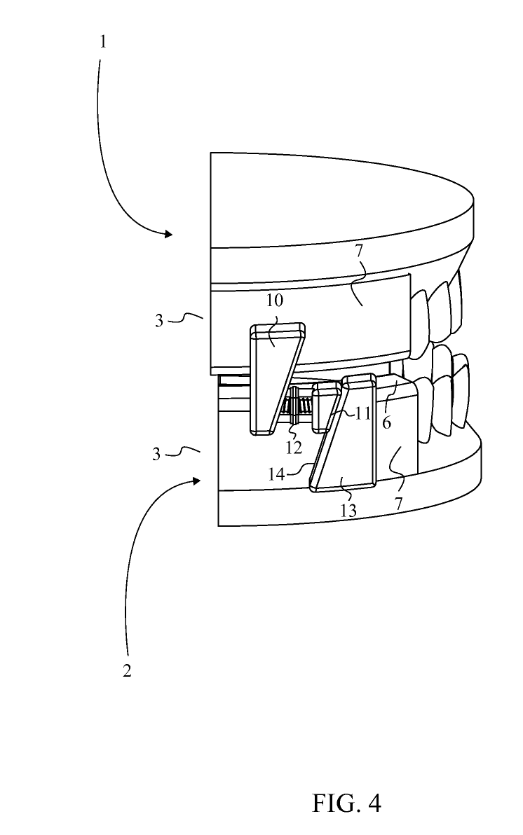

Referencing FIG. 4 and FIG. 6, the maxillary dental plate 1 comprises a pair of upper contact structures 10. The pair of upper contact structures 10 are a vertical protrusions coupled to the buccal surfaces 7 of the first side section 3 and the second side section 4, opposite the facial surfaces of the posterior teeth. The pair of upper contact structures 10 comprise a maxillary contact surface 11 and a surface advancement mechanism 12. The maxillary contact surface 11 is a forward moving surface which serves as the point of contact with the mandibular dental plate 2. Movement of the maxillary contact surface 11 provides the present invention with the ability to advance the mandibular dental plate 2. The forward movement to the maxillary contact surface 11 is provided by a surface advancement mechanism 12 that is expandably coupled to the maxillary contact surface 11. The surface advancement mechanism 12 is able to adjust the length of the forward movement of the maxillary contact surface 11 according to the desired need of a particular user. The maxillary dental plate 1 being removably coupled to the upper jaw remains stationary relative to the movement of the maxillary contact surface 11.

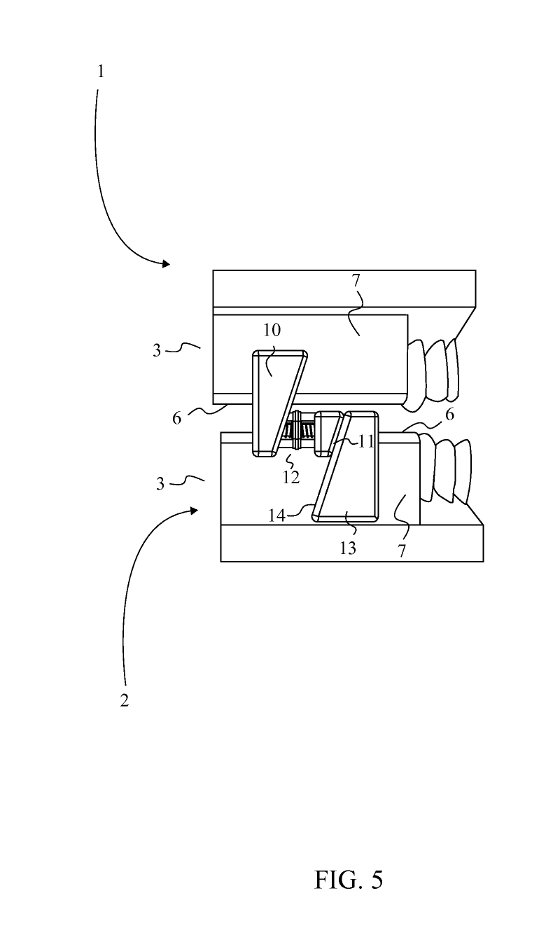

Referencing FIG. 5 and FIG. 7, mandibular dental plate 2 comprises a pair of lower contact structures 13. The pair of lower contact structures 13 are vertical protrusions coupled to the buccal surfaces 7 of the first side section 3 and the second side sections 4, opposite the facial surfaces of the posterior teeth. The pair of lower contact structures 13 each comprise a mandibular contact surface 14. The mandibular contact surface 14 is the point of contact between the maxillary contact surface 11 and the mandibular dental plate 2. When the maxillary contact surface 11 is advanced by the surface advancement mechanism 12, the mandibular contact surface 14 transfers the movement to the mandibular dental plate 2. The mandibular dental plate 2 being removably coupled to the posterior teeth of the user's lower jaw, is able to tighten the soft tissue and muscles localized around the airway, alleviating constriction to the trachea. Additionally, the forward positioning of the lower jaw advances the user's tongue forward preventing occlusion to the trachea.

In the present invention the anterior teeth for both the upper and the lower jaws are left uncovered on the incisal and the labial surfaces to provide a user with maximum comfort during use. The maxillary dental plate 1 and the mandibular dental plate 2 leave the incisal and labial surfaces of the central incisors, the lateral incisors, and the cuspids uncovered to provide a user with a comfortable amount of lingual space and with the ability to close a user's lips during use. These improvements to comfort during use provide the present invention with better patient compliance for the treatment. During use, the present invention moves the lower jaw and tongue forward by 6.5 mm-8 mm. This forward positioning is uncomfortable for some users, but the present invention is able to relieve this discomfort by excluding obstructions to the incisal and the labial surfaces. The lack of obstructions to the incisal and labial surfaces of the anterior teeth provides the users with additional lingual space within their mouth. Additionally, the present invention provides the users with approximately 7 mm of space between the incisal surfaces of the upper jaw and the incisal surfaces of the lower jaw. This distance between the upper and the lower jaw allows the user the ability to the close their lips during use which provides additional comfort and further ensure compliance with the treatment.

In the current embodiment of the present invention the lingual surface mount 5 is positioned against the lingual surfaces of the posterior and anterior teeth in order to disperse pressure from the mandibular advancement across a wider surface area. By dispersing the pressure applied to the incisal teeth across the lingual surface, the lingual surface mount 5 is able to reduce point localized pressure. The reduction of localized pressure points prevents the present invention from causing rotation and movement to the anterior teeth, which could potentially cause eruptions and deformations over extended use.

In the current embodiment of the present invention the maxillary dental plate 1 and the mandibular dental plate 2 are both custom fitted dental appliances that are formed using a clear acrylic. The formed clear acrylic is fitted to the lingual surfaces as well as the occlusal and buccal surfaces 7 of the posterior teeth. It should be noted that the acrylic layer covering the occlusal surfaces 6 of the posterior teeth is formed in a manner that decreases or eliminates the grinding and bruxing experienced by some users while utilizing dental appliances. While clear acrylic is the material currently used to construct the present invention, it should be understood as an obvious difference that additional materials could potentially be substituted or used in combination with the clear acrylic. Additional materials that could be used in the construction of the present invention include, but are not limited to, polypropylene derived materials, polyvinylchloride derived materials, and polyurethane derived materials as well as any material capable of being safely used in the construction of a dental appliance.

In the present invention the pair of posterior teeth connection methods 9 each comprise a pair of ball clasp couplers. The pair of ball clasp couplers are mounted between the second biscuspid and the first molar or second molar. while the current embodiment of the present invention utilizes ball clasp couplers as the preferred posterior teeth connection method it should be understood as an obvious difference to utilizes an alternative connection method that is capable of providing the same removable engagement while leaving the incisal surfaces and the labial surfaces of the anterior teeth uncovered. Furthermore, while the posterior teeth connections methods 9 are mounted between the second biscuspid and the first or second molar in the current embodiment of the present invention, it should be understood as an obvious difference that alternative positioning of the posterior teeth connection method 9 would be required to accommodate abnormal variations to the positioning of a user's teeth.

In the current embodiment of the present invention the surface advancement mechanism 12 allows the maxillary contact surface 11 to advance the mandibular dental plate 2 a distance between 6.5 mm to 8 mm. While this is the current maximum range the present invention can be adjusted to, it should be understood as an obvious difference to permit the surface advancement mechanism 12 to extend to ranges that are less than or greater than 6.5 mm and 8 mm, respectively. Furthermore it should be noted that the surface advancement mechanism 12 provided in the current embodiment of the present invention is most similar to an adjustable jack screw mechanism. While the current embodiment of the present invention utilizes an adjustable jack screw mechanism as the surface advancement mechanism 12 it should be noted that alternative mechanisms could potentially be utilized while providing the same functionality and should therefore be considered an obvious difference.

In the current embodiment of the present invention the vertical distance between the incisal teeth of the upper jaw and the incisal teeth of the lower jaw is maintained between 7 mm and 8 mm. The current distance utilized by the preferred embodiment of the present invention is provided to allow a maximum opening between the upper and lower jaw that provides maximum lingual comfort and the ability for a user to close their lips. The distance between incisal teeth is a measure from the incisal surface of the anterior maxillary teeth to the incisal surface of the anterior mandibular teeth. While the current embodiment has the distance between the incisal teeth of the upper and lower jaw set between the range of 7 mm and 8 mm, it should be understood as an obvious difference to provide variations to the distance between the upper and lower jaw due to known variations in the size and height of individuals fitted for the present invention.

Although the invention has been explained in relation to its preferred embodiment, it is to be understood that many other possible modifications and variations can be made without departing from the spirit and scope of the invention as hereinafter claimed.

What is claimed is:

1. An oral sleep apnea device provided for advancing the lower jaw of a user while providing increased lingual comfort by eliminating direct contact with the incisal surface and the labial surfaces of the anterior teeth during use comprises,
a maxillary dental plate;
a mandibular dental plate;
both the maxillary dental plate and the mandibular dental plate comprise a first side section, a second side section, and a lingual surface mount;
the maxillary dental plate comprises a pair of upper contact structures;
the mandibular dental plate comprises a pair of lower contact structures;
the first side section and the second side section each comprise an occlusal surface and a buccal surface;
the lingual surface mount comprises a lingual splint and a pair of posterior teeth connection methods;
the pair of upper contact structures each comprise an maxillary contact surface and a surface advancement mechanism; and
the pair of lower contact structures each comprise a mandibular contact surface.

2. The oral sleep apnea device provided for advancing the lower jaw of a user while providing increased lingual comfort by eliminating direct contact with the incisal surface and the labial surfaces of the anterior teeth during use as claimed in claim 1 comprises:
both the maxillary dental plate and the mandibular dental plate being arcuately shaped;
both the maxillary dental plate and the mandibular dental plate being bilaterally symmetrical across a median line, wherein the median line is an imaginary line which traverse from the superior position to the inferior position bisecting a user's jaw;
the maxillary dental plate being positioned above the mandibular dental plate; and
the mandibular dental plate being extendably coupled adjacent the maxillary dental plate.

3. The oral sleep apnea device provided for advancing the lower jaw of a user while providing increased lingual comfort by eliminating direct contact with the incisal surface and the labial surfaces of the anterior teeth during use as claimed in claim 1 comprises:
the first side section and the second side section being adjacently coupled to the lingual surface mount; and
the first side section being positioned opposite the second side section across a median line, wherein the median line is an imaginary line which traverse from the superior position to the inferior position bisecting a user's jaw.

4. The oral sleep apnea device provided for advancing the lower jaw of a user while providing increased lingual comfort by eliminating direct contact with the incisal surface and the labial surfaces of the anterior teeth during use as claimed in claim 1 comprises:
the lingual surface mount being attached to the occlusal surface of the first side section and to the occlusal surface of the second side section;
the lingual splint being positioned within the lingual surface mount; and
the pair of posterior teeth connection methods being coupled adjacent to the lingual splint.

5. The oral sleep apnea device provided for advancing the lower jaw of a user while providing increased lingual comfort by eliminating direct contact with the incisal surface and the labial surfaces of the anterior teeth during use as claimed in claim 1 comprises:
Each posterior teeth connection method of the pair of posterior teeth connection methods being housed by either the first side section or the second side section, wherein the posterior teeth connection method being surrounded by the structural relationship formed by the buccal surface and the occlusal surface to the lingual surface mount.

6. The oral sleep apnea device provided for advancing the lower jaw of a user while providing increased lingual comfort by eliminating direct contact with the incisal surface and the labial surfaces of the anterior teeth during use as claimed in claim 1 comprises:
the upper contact structure being operatively engaged with the lower contact structure;
the pair of upper contact structures being affixed to the buccal surfaces of the first side section and the second side section of the maxillary dental plate, wherein each of the upper contact structures being coupled to either the first side section or the second side section; and
the pair of lower contact structures being affixed to the buccal surfaces of the first side section and the second side section of the mandibular dental plate, wherein each of the lower contact structures being coupled to either the first side section or the second side section.

7. The oral sleep apnea device provided for advancing the lower jaw of a user while providing increased lingual comfort by eliminating direct contact with the incisal surface and the labial surfaces of the anterior teeth during use as claimed in claim 1 comprises:
The maxillary contact surface being coincident with the mandibular contact surface, opposite the surface advancement mechanism; and
the maxillary contact surface being expandably coupled to the surface advancement mechanism.

8. An oral sleep apnea device provided for advancing the lower jaw of a user while providing increased lingual comfort by eliminating direct contact with the incisal surface and the labial surfaces of the anterior teeth during use comprises,
a maxillary dental plate;
a mandibular dental plate;
both the maxillary dental plate and the mandibular dental plate comprise a first side section, a second side section, and a lingual surface mount;
the maxillary dental plate comprises a pair of upper contact structures;
the mandibular dental plate comprises a pair of lower contact structures;
the first side section and the second side section each comprise an occlusal surface and a buccal surface;
the lingual surface mount comprises a lingual splint and a pair of posterior teeth connection methods;
the pair of upper contact structures each comprise an maxillary contact surface and a surface advancement mechanism;
the pair of lower contact structures each comprises a mandibular contact surface;
both the maxillary dental plate and the mandibular dental plate being arcuately shaped;
both the maxillary dental plate and the mandibular dental plate being bilaterally symmetrical across a median line, wherein the median line is an imaginary line which traverse from the superior position to the inferior position bisecting a user's jaw;
the maxillary dental plate being positioned above the mandibular dental plate; and
the mandibular dental plate being extendably coupled adjacent the maxillary dental plate.

9. The oral sleep apnea device provided for advancing the lower jaw of a user while providing increased lingual comfort by eliminating direct contact with the incisal surface and the labial surfaces of the anterior teeth during use as claimed in claim 8 comprises:
the first side section and the second side section being adjacently coupled to the lingual surface mount; and
the first side section being positioned opposite the second side section across a median line, wherein the median line is an imaginary line which traverse from the superior position to the inferior position bisecting a user's jaw.

10. The oral sleep apnea device provided for advancing the lower jaw of a user while providing increased lingual comfort by eliminating direct contact with the incisal surface and the labial surfaces of the anterior teeth during use as claimed in claim 8 comprises:
the lingual surface mount being attached to the occlusal surface of the first side section and to the occlusal surface of the second side section;
the lingual splint being positioned within the lingual surface mount; and
the pair of posterior teeth connection methods being coupled adjacent to the lingual splint.

11. The oral sleep apnea device provided for advancing the lower jaw of a user while providing increased lingual comfort by eliminating direct contact with the incisal surface and the labial surfaces of the anterior teeth during use as claimed in claim 8 comprises:
Each posterior teeth connection method of the pair of posterior teeth connection methods being housed by either the first side section or the second side section, wherein the posterior teeth connection method being surrounded by the structural relationship formed by the buccal surface and the occlusal surface to the lingual surface mount.

12. The oral sleep apnea device provided for advancing the lower jaw of a user while providing increased lingual comfort by eliminating direct contact with the incisal surface and the labial surfaces of the anterior teeth during use as claimed in claim 8 comprises:
the upper contact structure being operatively engaged with the lower contact structure;
the pair of upper contact structures being affixed to the buccal surfaces of the first side section and the second side section of the maxillary dental plate, wherein each of the upper contact structures being coupled to either the first side section or the second side section; and
the pair of lower contact structures being affixed to the buccal surfaces of the first side section and the second side section of the mandibular dental plate, wherein each of the lower contact structures being coupled to either the first side section or the second side section.

13. The oral sleep apnea device provided for advancing the lower jaw of a user while providing increased lingual comfort by eliminating direct contact with the incisal surface and the labial surfaces of the anterior teeth during use as claimed in claim 8 comprises:
The maxillary contact surface being coincident with the mandibular contact surface, opposite the surface advancement mechanism; and
the maxillary contact surface being expandably coupled to the surface advancement mechanism.

14. An oral sleep apnea device provided for advancing the lower jaw of a user while providing increased lingual comfort by eliminating direct contact with the incisal surface and the labial surfaces of the anterior teeth during use comprises,
a maxillary dental plate;
a mandibular dental plate;
both the maxillary dental plate and the mandibular dental plate comprise a first side section, a second side section, and a lingual surface mount;
the maxillary dental plate comprises a pair of upper contact structures;
the mandibular dental plate comprises a pair of lower contact structures;
the first side section and the second side section each comprise an occlusal surface and a buccal surface;
the lingual surface mount comprises a lingual splint and a pair of posterior teeth connection methods;
the pair of upper contact structures each comprise an maxillary contact surface and a surface advancement mechanism; and
the pair of lower contact structures each comprises a mandibular contact surface.
both the maxillary dental plate and the mandibular dental plate being arcuately shaped;
both the maxillary dental plate and the mandibular dental plate being bilaterally symmetrical across a median line, wherein the median line is an imaginary line which traverse from the superior position to the inferior position bisecting a user's jaw;
the maxillary dental plate being positioned above the mandibular dental plate; and
the mandibular dental plate being extendably coupled adjacent the maxillary dental plate.

15. The oral sleep apnea device provided for advancing the lower jaw of a user while providing increased lingual comfort by eliminating direct contact with the incisal surface and the labial surfaces of the anterior teeth during use as claimed in claim 14 comprises:
the first side section and the second side section being adjacently coupled to the lingual surface mount;
the first side section being positioned opposite the second side section across a median line, wherein the median line is an imaginary line which traverse from the superior position to the inferior position bisecting a user's jaw;
the lingual surface mount being attached to the occlusal surface of the first side section and to the occlusal surface of the second side section;
the lingual splint being positioned within the lingual surface mount; and
the pair of posterior teeth connection methods being coupled adjacent to the lingual splint.

16. The oral sleep apnea device provided for advancing the lower jaw of a user while providing increased lingual comfort by eliminating direct contact with the incisal surface and the labial surfaces of the anterior teeth during use as claimed in claim 14 comprises:
Each posterior teeth connection method of the pair of posterior teeth connection methods being housed by either the first side section or the second side section, wherein the posterior teeth connection method being surrounded by the structural relationship formed by the buccal surface and the occlusal surface to the lingual surface mount.

17. The oral sleep apnea device provided for advancing the lower jaw of a user while providing increased lingual comfort by eliminating direct contact with the incisal surface and the labial surfaces of the anterior teeth during use as claimed in claim 14 comprises:
the upper contact structure being operatively engaged with the lower contact structure;
the pair of upper contact structures being affixed to the buccal surfaces of the first side section and the second side section of the maxillary dental plate, wherein each of the upper contact structures being coupled to either the first side section or the second side section;
the pair of lower contact structures being affixed to the buccal surfaces of the first side section and the second side section of the mandibular dental plate, wherein each of the lower contact structures being coupled to either the first side section or the second side section;
The maxillary contact surface being coincident with the mandibular contact surface, opposite the surface advancement mechanism; and
the maxillary contact surface being expandably coupled to the surface advancement mechanism.
